(19) United States
(12) Patent Application Publication
(10) Pub. No.: US 2020/0207848 A1
(43) Pub. Date: Jul. 2, 2020

(54) USE OF IL-1B BINDING ANTIBODIES FOR THE TREATMENT OF ALCOHOLIC HEPATITIS

(71) Applicant: NOVARTIS AG, Basel (CH)

(72) Inventor: Shephard MPOFU, Oberwil (CH)

(21) Appl. No.: 16/646,608

(22) PCT Filed: Sep. 11, 2018
(86) PCT No.: PCT/IB2018/056928

§ 371 Date: Mar. 12, 2020

Related U.S. Application Data
(60) Provisional application No. 62/557,929, filed on Sep. 13, 2017.
(60) Provisional application No. 62/647,088, filed on Mar. 23, 2018.

(51) Int. Cl. C07K 16/24; A61P 1/16

(52) U.S. Cl. CPC C07K 16/245; A61K 2039/545; A61P 1/16

(57) ABSTRACT

The present invention relates to a method for treating or alleviating the symptoms of alcoholic hepatitis in a subject, comprising administering gevokizumab or 2-5 mg of canakinumab per kg of body weight to a subject in need thereof.

### FIELD OF THE INVENTION

The present disclosure relates to a novel use and dosage regimens of the IL-Iβ binding antibodies canakinumab and gevokizumab, for treating or alleviating the symptoms of alcoholic hepatitis.

### BACKGROUND OF THE INVENTION

Excessive alcohol use is a major cause of liver disease in the Western world. Although it is not fully understood how alcohol damages the liver, chronic alcohol consumption results in the secretion of pro-inflammatory cytokines, oxidative stress, lipid peroxidation, and acetaldehyde toxicity, resulting in inflammation, apoptosis and eventually fibrosis of liver cells. The three most widely recognized steps of alcoholic liver disease are alcoholic fatty liver or alcoholic hepatic steatosis, alcoholic hepatitis and alcoholic cirrhosis. At least 80% of heavy drinkers develop steatosis, 10-35% develop alcoholic hepatitis and approximately 10% develop cirrhosis. Alcoholic hepatic steatosis, also called alcoholic fatty liver, consists in the occupation of a large proportion of the cytoplasm of affected hepatocytes by a single large triglyceride occlusion. This state is reversible by abstinence but may progress to cirrhosis if excess alcohol intake persists. Alcoholic hepatitis is the second main step of alcoholic liver disease and associates steatosis together with inflammation and necrosis, due to excessive intake of alcohol, and carries with it a significant mortality risk. Alcoholic cirrhosis is the most severe and terminal step of alcoholic liver disease and is characterized by fibrosis, leading to a progressive loss of liver function.

IL-1β is a pro-inflammatory cytokine produced by a variety of cell types, particularly mononuclear phagocytes, in response to injury, infection and inflammation. In alcoholic liver disease, IL-1β has been shown to be an important contributor to hepatic inflammation, leading to metabolic disturbances, fibrogenesis, stellate cell activation and portal hypertension. Thus, IL-1β represents a potential therapeutic target for treating or alleviating the symptoms of alcoholic hepatitis.

At present, recommended treatment options for alcoholic hepatitis include prednisolone, but this option carries with it a risk of higher incidence of infection, resulting in no survival advantage at 90 days.

Thus, there is an urgent need for new methods of preventing and/or ameliorating the effects of alcoholic hepatitis.

### SUMMARY OF THE INVENTION

Accordingly, in one aspect, the present invention is directed to a method of treating or alleviating the symptoms of alcoholic hepatitis in a subject, comprising administering of 2-5 mg canakinumab per kg body weight to the subject.

In another aspect, the present invention is directed to canakinumab for use in treating or alleviating the symptoms of alcoholic hepatitis in a subject, comprising administering of 2-5 mg canakinumab per kg body weight to the subject.

In yet another aspect, the present invention is directed to the use of canakinumab for the manufacture of a medicament for treating or alleviating the symptoms of alcoholic hepatitis in a subject, comprising administering of 2-5 mg canakinumab per kg body weight to the subject.

The present invention is also directed to a method of treating or alleviating the symptoms of alcoholic hepatitis in a subject, comprising administering gevokizumab to the subject.

In another aspect, the present invention is directed to gevokizumab for use as a medicament for treating or alleviating the symptoms of alcoholic hepatitis in a subject, comprising administering gevokizumab to the subject.

In yet another aspect, the present invention is directed to the use of gevokizumab for the manufacture of a medicament for treating or alleviating the symptoms of alcoholic hepatitis in a subject, comprising administering gevokizumab to the subject.

Further features and advantages of the invention will become apparent from the following description.

### DETAILED DESCRIPTION OF THE INVENTION

Alcoholic hepatitis can range from mild hepatitis, with abnormal laboratory tests being the only indication of disease, to severe liver dysfunction with complications such as jaundice (yellow skin caused by bilirubin retention), hepatic encephalopathy (neurological dysfunction caused by liver failure), ascites (fluid accumulation in the abdomen), bleeding esophageal varices (varicose veins in the esophagus), abnormal blood clotting and coma. Alcoholic hepatitis is reversible if the patient stops drinking, but hepatitis usually takes several months to resolve. Alcoholic hepatitis can lead to liver scarring and cirrhosis. The typical findings on liver histology include hepatocellular necrosis and ballooning degeneration, and alcoholic Mallory's hyaline bodies (abnormal aggregations of cellular intermediate filament proteins indicative of fibrosis). Cholestasis is prominent. Severity of the disease can be classified according to Maddrey's discriminant function (mDF) (based on bilirubin and prothrombin time), Glasgow alcoholic hepatitis score (based on age, white blood cell count, urea, prothrombin time and bilirubin) or Model for End Stage Liver Disease (MELD) score (based on creatinine, bilirubin and INR) (Lucey et al. (2009) N. Engl. J. Med., 360(26), 2758-2769; Vergis et al. (2017) Gastroenterology. 2017 April; 152(5):1068-1077.e4). Alcoholic hepatitis is classified as severe when the mDF is ≥32.

The present invention provides, inter alia, methods of treating or alleviating the symptoms of alcoholic hepatitis in a subject, comprising administering of 2-5 mg canakinumab per kg body weight to the subject.

Canakinumab (international nonproprietary name (INN) number 8836) is disclosed in WO02/16436, which is hereby incorporated by reference in its entirety. Canakinumab is a fully human monoclonal anti-human IL-1β antibody of the IgG1/k isotype, and is approved under the trade name Ilaris® for the treatment of IL-1β driven inflammatory diseases. It is designed to bind to human IL-1β, and thereby blocking the interaction of the cytokine with its receptors. The antagonism of IL-1β mediated inflammation using canakinumab in lowering high sensitivity C-reactive protein (hsCRP) and other inflammatory marker levels has shown an acute phase response in patients with Cryopyrin-Associated Periodic Syndrome (CAPS) and rheumatoid arthritis.

Also provided is a method of treating or alleviating the symptoms of alcoholic hepatitis in a subject, comprising administering gevokizumab to the subject.

Gevokizumab (XOMA-052) is a high-affinity, humanized monoclonal antibody of the IgG2 isotype to interleukin-1β, developed for the treatment of IL-1β driven inflammatory diseases. Gevokizumab modulates IL-1β binding to its signaling receptor. Gevokizumab is disclosed in WO2007/002261, which is hereby incorporated by reference in its entirety.

Alcoholic hepatitis is characterized by elevated bilirubin, which reflects impaired metabolic function of the liver in the absence of biliary obstruction. Accordingly, one embodiment is the use of canakinumab in treating or alleviating the symptoms of alcoholic hepatitis in a subject with serum bilirubin levels of >80 μmol/L before administration of canakinumab. Another embodiment is the use of gevokizumab in treating or alleviating the symptoms of alcoholic hepatitis in a subject with serum bilirubin levels of >80 μmol/L before administration of gevokizumab.

Accordingly, a decrease in serum bilirubin levels indicates recovery of metabolic function of the liver. One embodiment is the use of canakinumab in treating or alleviating the symptoms of alcoholic hepatitis in a subject with serum bilirubin baseline levels of >80 μmol/L before administration of canakinumab and a decrease of >25% of serum bilirubin compared to baseline at least 7 days after first administration of canakinumab. Another embodiment is the use of gevokizumab in treating or alleviating the symptoms of alcoholic hepatitis in a subject with serum bilirubin baseline levels of >80 μmol/L before administration of gevokizumab and a decrease of >25% of serum bilirubin compared to baseline at least 7 days after first administration of gevokizumab.

Excess alcohol intake over many years can lead to alcoholic liver disease and alcoholic hepatitis. As used herein, excess alcohol intake is characterized by alcohol intake of >80 g/day for males or >60 g/day for females. Accordingly, one embodiment is the use of canakinumab in treating or alleviating the symptoms of alcoholic hepatitis in a male or female subject consuming excess alcohol, wherein said male subject is consuming>80 g of alcohol per day or said female subject is consuming>60 g of alcohol per day. Another embodiment is the use of gevokizumab in treating or alleviating the symptoms of alcoholic hepatitis in a male or female subject consuming excess alcohol, wherein said male subject is consuming>80 g of alcohol per day or said female subject is consuming>60 g of alcohol per day.

The Model for End-Stage Liver Disease (MELD) is a scoring system for assessing the severity of chronic liver disease. MELD uses the patient's values for serum bilirubin, serum creatinine, and the international normalized ratio for prothrombin time (INR) to predict survival and is calculated according to the formula:

MELD=3.78×ln[serum bilirubin (mg/dL)]+11.2×ln[INR]+9.57×ln[serum creatinine (mg/dL)]+6.43

TABLE 1
3-Month Mortality Based on MELD Scores:

| MELD Score | Mortality Probability |
|---|---|
| 40 | 71.3% mortality |
| 30-39 | 52.6% mortality |
| 20-29 | 19.6% mortality |
| 10-19 | 6.0% mortality |
| 9 or less | 1.9% mortality |

Accordingly, one embodiment is the use of canakinumab in treating or alleviating the symptoms of alcoholic hepatitis in a subject, wherein said subject is characterized by Model for End-Stage Liver Disease (MELD) score of ≤25 before administration of canakinumab. Another embodiment is the use of gevokizumab in treating or alleviating the symptoms of alcoholic hepatitis in a subject, wherein said subject is characterized Model for End-Stage Liver Disease (MELD) score of ≤25 before administration of gevokizumab.

The Maddrey discriminant function (mDF) is a model for evaluating the severity and prognosis in alcoholic hepatitis and evaluates the efficacy of steroid treatment in alcoholic hepatitis. The mDF score is a statistical model useful for predicting a subject's short term prognosis, in particular mortality within 30 or 90 days. A score of 32 or greater implies poor outcome with 30 day mortality ranging between 35% to 45%. The mDF is calculated according to the formula:

mDF=4.6×(Prothrombin time (PT_{PATIENT}−PT_{CONTROL})+Serum Bilirubin (μmol/l)/17.1

Accordingly, one embodiment to the use of canakinumab in treating or alleviating the symptoms of alcoholic hepatitis in a subject, wherein said subject is characterized by Maddrey discriminant function (mDF) score of ≥32 before administration of canakinumab. Another embodiment is the use of gevokizumab in treating or alleviating the symptoms of alcoholic hepatitis in a subject, wherein said subject is characterized by Maddrey discriminant function (mDF) score of ≥32 before administration of gevokizumab.

Accordingly, in one embodiment provided is the use of canakinumab in treating or alleviating the symptoms of alcoholic hepatitis in a subject, wherein said subject is characterized by Maddrey discriminant function (mDF) score≥32 and Model for End-Stage Liver Disease (MELD) score of ≤25 before administration of canakinumab. In another embodiment provided is the use of gevokizumab in treating or alleviating the symptoms of alcoholic hepatitis in a subject, wherein said subject is characterized by Maddrey discriminant function (mDF) score of ≥32 and Model for End-Stage Liver Disease (MELD) score of ≤25 before administration of gevokizumab.

In one embodiment provided is canakinumab for use is in treating or alleviating the symptoms of alcoholic hepatitis in a subject, wherein said subject has reduced risk of mortality 90 days after first administration of canakinumab compared to a subject not receiving canakinumab. In one embodiment provided is gevokizumab for use is in treating or alleviating the symptoms of alcoholic hepatitis in a subject, wherein said subject has reduced risk of mortality 90 days after first administration of gevokizumab compared to a subject not receiving gevokizumab.

The Glasgow Alcoholic Hepatitis Score (GAHS) can be used to identify patients at risk of mortality (Forrest et al. (2007) Gut, 56:1743-1746). A score is given for each parameter according to the following table and a total score is calculated. A score of 9 or more identify patients most at risk of death.

TABLE 2
Glasgow Alcoholic Hepatitis Scoring (GAHS) system

| Score given | 1 | 2 | 3 |
|---|---|---|---|
| Age | <50 | ≥50 | — |
| White cell count (WCC) | <15 | ≥15 | — |
| (10⁹/L) | | | |
| Urea (mmol/L) | <5 | ≥5 | — |
| Prothrombin time (PT) | <1.5 | 1.5-2.0 | >2.0 |
| ratio or International | | | |
| normalised ratio (INR) | | | |
| Bilirubin (μmol/L) | <125 | 125-250 | >250 |

The Glasgow Alcoholic Hepatitis Score (GAHS) is correlated with survival rates as detailed in the following table:

TABLE 3
GAHS and survival rates

| | Day 28 survival (%) | Day 84 survival (%) |
|---|---|---|
| Day 1 score | | |
| GAHS <9 | 87 | 79 |
| GAHS ≥9 | 46 | 40 |
| Day 6-9 score | | |
| GAHS <9 | 93 | 86 |
| GAHS ≥9 | 47 | 37 |

Accordingly, it is one aspect of the invention to improve the GAHS score. One embodiment is the use of canakinumab in treating or alleviating the symptoms of alcoholic hepatitis in a subject, wherein said subject is characterized by lowered GAHS score at least 7 days, at least 14 days, at least 21 days, at least 28 days, at least 42 days or at least 90 days compared to before first administration of canakinumab. Another embodiment is the use of canakinumab in treating or alleviating the symptoms of alcoholic hepatitis in a subject, comprising administering 2-5 mg canakinumab per kg body weight of the subject and wherein said subject is characterized by lowered GAHS score at least 7 days, at least 14 days, at least 21 days, at least 28 days, at least 42 days or at least 90 days compared to before first administration of canakinumab. Another embodiment is the use of gevokizumab in treating or alleviating the symptoms of alcoholic hepatitis in a subject, wherein said subject is characterized by lowered GAHS score at least 7 days, at least 14 days, at least 21 days, at least 28 days, at least 42 days or at least 90 days compared to before first administration of gevokizumab.

The Lille score predicts mortality in patients with alcoholic hepatitis, which are not responding to first-line steroid therapy. The Lille score is calculated according to the following formula:

Exp(−R)/[1+exp(−R)]

where
R=[3.19−(0.101*age in years)]+(1.47*albumin at baseline in g/dL)+[0.28215*(bilirubin at baseline−bilirubin at Day 8 in mg/dL)]−[0.206*(if creatinine>=1.3 mg/dL at baseline)]−[0.11115*bilirubin baseline in mg/dL]−(0.0096*Prothrombin Time in seconds at baseline)

Accordingly, it is one aspect of the invention to improve the Lille score. One embodiment is the use of canakinumab in treating or alleviating the symptoms of alcoholic hepatitis in a subject and wherein said subject is characterized by lowered Lille score at least 7 days compared to before first administration of canakinumab. Another embodiment is the use of canakinumab in treating or alleviating the symptoms of alcoholic hepatitis in a subject, comprising administering 2-5 mg canakinumab per kg body weight of the subject and wherein said subject is characterized by lowered Lille score at least 7 days compared to before first administration of canakinumab. In yet another embodiment provided is the use of gevokizumab in treating or alleviating the symptoms of alcoholic hepatitis in a subject, comprising administering gevokizumab, and wherein said subject is characterized by lowered Lille score at least 7 days compared to before first administration of gevokizumab.

Renal dysfunction is a common complication of liver injury and can lead to acute kidney injury (AKI). Acute kidney injury is defined as
Rise in blood creatinine by 26 micromoles per liter or more within 48 hours
Rise in blood creatinine over time by 50% or more within the past 7 days or
Urine output than 0.5 ml per kg per hour for more than 6 hours

It is one aspect of the invention to decrease the risk of acute kidney injury in a subject with alcoholic hepatitis. Accordingly, one embodiment is the use of canakinumab in treating or alleviating the symptoms of alcoholic hepatitis in a patient and wherein said subject has decreased risk of suffering from acute kidney injury within 90 days of first administration of canakinumab. Another embodiment is the use of canakinumab in treating or alleviating the symptoms of alcoholic hepatitis in a patient, comprising administering 2-5 mg canakinumab per kg body weight of the subject and wherein said subject has decreased risk of suffering from acute kidney injury within 90 days of first administration of canakinumab. In another embodiment provided is the use of gevokizumab in treating or alleviating the symptoms of alcoholic hepatitis in a patient, comprising administering gevokizumab to the subject and wherein said subject has decreased risk of suffering from acute kidney injury within 90 days of first administration of gevokizumab.

Liver injury can be assessed by liver biopsy, which may be obtained via transcutaneous or transjugular route depending on the patient's coagulation status. Lobular inflammation may be assessed. Portal tracts or equivalents may be assessed for polymorphonuclear cell infiltrate, ballooned hepatocytes and/or steatosis. Scoring systems evaluating histology from liver biopsies may be used to assess prognosis of alcoholic hepatitis patients, in particular 90 day mortality. Suitable scoring systems are the Alcoholic Hepatitis Histologic Score (AHHS) (Altamirano et al, (2014) Gastroenterology., 146(5), 1231-9.e1-6) and the Nonalcoholic Fatty Liver Disease (NAFLD) Activity Score (NAS).

It is one aspect of the invention to decrease inflammation of the liver in a subject with alcoholic hepatitis. In one embodiment, histological improvement of alcoholic hepatitis is characterized by reduction in lobular inflammation assessed after at least 4 weeks (28 days) from first administration of canakinumab. In another embodiment, histological improvement of alcoholic hepatitis is characterized by reduction in lobular inflammation assessed after at least 4 weeks (28 days) from first administration of gevokizumab. In certain aspects of the invention, a histological improvement of alcoholic hepatitis is characterized by resolution of individual components of alcoholic hepatitis such as polymorphonuclear cell infiltrate, ballooned hepatocytes and/or steatosis in liver cells and is detected by liver biopsy at least 4 weeks (28 days) after first administration of canakinumab or after first administration of gevokizumab. In one embodiment provided is canakinumab for use in treating or alleviating the symptoms of alcoholic hepatitis in a patient, comprising administering 2-5 mg canakinumab per kg body weight of the subject and wherein said subject has histological improvement of the liver assessed at least 4 weeks (28 days) after first administration of canakinumab. In another embodiment, provided is gevokizumab for use in treating or alleviating the symptoms of alcoholic hepatitis in a patient, comprising administering gevokizumab to the subject and wherein said subject has histological improvement of the liver assessed at least 4 weeks (28 days) after first administration of gevokizumab. Said histological improvement may comprise reduction in lobular inflammation. In an alternative or additional embodiment, said histological improvement may also comprise resolution of individual components of alcoholic hepatitis, such as polymorphonuclear cell infiltrate, ballooned hepatocytes and/or steatosis.

Previous studies with anti-cytokine therapy in alcoholic hepatitis, targeting the TNF-α system, have failed to show a survival benefit due to the increased risk of infection and possibly due to the removal of a regenerative signal provided by TNF-α (Boetticher et al. (2008) Gastroenterology, 135(6):1953-60). The overall risk of infection in alcoholic hepatitis is 20-30% but this is increased 3-4 fold in patients treated with immunosuppressive drugs such as prednisolone when the pre-treatment bacterial 16S ribosomal DNA (16S rDNA) in blood is >18.5 pg/ml (Vergis et al., 2017). Accordingly, subjects at high risk of bacterial infection may receive prophylactic antibiotics. Herein, high risk of bacterial infection is characterized by levels of 16S ribosomal DNA (16S rDNA) in blood of >18.5 pg/ml.

Accordingly, in one embodiment canakinumab is used in treating or alleviating the symptoms of alcoholic hepatitis in a subject, wherein antibiotics are administered for at least 14 days following first administration of canakinumab and wherein the subject is at high risk of bacterial infection characterized by levels of 16S ribosomal DNA (16S rDNA) in the blood of >18.5 pg/ml. Another embodiment provides canakinumab for use for treating or alleviating the symptoms of alcoholic hepatitis in a subject, wherein antibiotics are administered for at least 14 days following first administration of canakinumab. In another embodiment provided is gevokizumab for use in treating or alleviating the symptoms of alcoholic hepatitis wherein antibiotics are administered for at least 14 days following first administration of gevokizumab and wherein the subject is at high risk of bacterial infection characterized by levels of 16S ribosomal DNA (16S rDNA) in the blood of >18.5 pg/ml. Another embodiment provides gevokizumab for use in treating or alleviating the symptoms of alcoholic hepatitis in a subject, wherein antibiotics are administered for at least 14 days following first administration of gevokizumab. Suitable antibiotics may comprise co-amoxyclav or ciprofloxacin.

Severe alcoholic hepatitis has high mortality and corticosteroids have been the mainstay of treatment for decades. Liver transplant can potentially provide long term benefit for patients, for example those which are steroid non-responders. In one aspect of the invention, canakinumab or gevokizumab are used for for treating or alleviating the symptoms of alcoholic hepatitis, resulting in reduced incidence of liver transplantation.

In one aspect of the invention canakinumab is used for treating or alleviating the symptoms of alcoholic hepatitis. In certain embodiments, canakinumab is administered at about 2-5 mg per kg body weight or about 3-5 mg per kg body weight or about 2-4 mg per kg body weight to a subject for treating or alleviating the symptoms of alcoholic hepatitis. In another embodiment, canakinumab is used for treating or alleviating the symptoms of alcoholic hepatitis in a subject, comprising administering 2-5 mg canakinumab per kg body weight of the subject. In another embodiment, canakinumab is used for treating or alleviating the symptoms of alcoholic hepatitis in a subject, comprising administering 2 mg canakinumab per kg body weight of the subject. In another embodiment, canakinumab is used for treating or alleviating the symptoms of alcoholic hepatitis in a subject, comprising administering 3 mg canakinumab per kg body weight of the subject. In another embodiment, canakinumab is used for treating or alleviating the symptoms of alcoholic hepatitis in a subject, comprising administering 4 mg canakinumab per kg body weight of the subject. In another embodiment, canakinumab is used for treating or alleviating the symptoms of alcoholic hepatitis in a subject, comprising administering 5 mg canakinumab per kg body weight of the subject. In different embodiments of the invention, canakinumab or gevokizumab can be administered parentally, e.g., intravenously or subcutaneously. Suitably, canakinumab or gevokizumab is administered intravenously to minimize the time to reach peak serum levels of the antibody. Alternatively, a dose of about 150 mg to about 600 mg or about 200 mg to about 600 mg or about 300 mg to about 600 mg or about 450 mg to about 600 mg of canakinumab may be administered subcutaneously for treating or alleviating the symptoms of alcoholic hepatitis in a subject. Alternatively, a dose of up to 600 mg of canakinumab may be administered subcutaneously for treating or alleviating the symptoms of alcoholic hepatitis in a subject.

In alcoholic liver disease patients, including in subjects with alcoholic hepatitis, levels of aspartate transaminase (AST) are generally elevated, and are indicative of liver cell injury. Thus, in one embodiment provided is the use of canakinumab in treating or alleviating the symptoms of alcoholic hepatitis in a patient, comprising administering at least one additional dose of 2-5 mg canakinumab per kg body weight of said patient, provided said patient has aspartate transaminase (AST) greater than twice upper limit of normal (ULN), wherein administration of the initial dose and additional dose of canakinumab are separated in time by at least four weeks (28 days). One embodiment comprises administering at least one additional dose of 3 mg canakinumab per kg body weight of said patient, provided said patient has aspartate transaminase (AST) greater than twice upper limit of normal (ULN), wherein administration of the initial dose and additional doses are separated in time by at least four weeks (28 days). Said initial and additional dose of canakinumab may be administered subcutaneously. Said initial dose of canakinumab may be administered intravenously, and said additional dose of canakinumab may be administered subcutaneously. Said initial and additional dose of canakinumab may be administered intravenously.

Another embodiment provides the use of gevokizumab in treating or alleviating the symptoms of alcoholic hepatitis in a patient, comprising administering at least one additional dose of gevokizumab to said patient, provided said patient has aspartate transaminase (AST) greater than twice upper limit of normal (ULN), wherein administration of the initial dose and additional dose of gevokizumab are separated in time by at least four weeks (28 days). Said initial and additional dose of gevokizumab may be administered subcutaneously. Said initial dose of gevokizumab may be administered intravenously, and said additional dose of gevokizumab may be administered subcutaneously. Said initial and additional dose of gevokizumab may be administered intravenously.

Canakinumab can be administered in a reconstituted formulation comprising canakinumab at a concentration of 50-200 mg/ml, 50-300 mM sucrose, 10-50 mM histidine, and 0.01-0.1% surfactant and wherein the pH of the formulation is 5.5-7.0. Canakinumab can be administered in a reconstituted formulation comprising canakinumab at a concentration of 50-200 mg/ml, 270 mM sucrose, 30 mM histidine and 0.06% polysorbate 20 or 80, wherein the pH of the formulation is 6.5.

Canakinumab can also be administered in a liquid formulation comprising canakinumab at a concentration of 50-200 mg/ml, a buffer system selected from the group consisting of citrate, histidine and sodium succinate, a stabilizer selected from the group consisting of sucrose, mannitol, sorbitol, arginine hydrochloride, and a surfactant and wherein the pH of the formulation is 5.5-7.0. Canakinumab can also be administered in a liquid formulation comprising canakinumab at a concentration of 50-200 mg/ml, 50-300 mM mannitol, 10-50 mM histidine and 0.01-0.1% surfactant, and wherein the pH of the formulation is 5.5-7.0. Canakinumab can also be administered in a liquid formulation comprising canakinumab at a concentration of 50-200 mg/ml, 270 mM mannitol, 20 mM histidine and 0.04% polysorbate 20 or 80, wherein the pH of the formulation is 6.5.

### Further Embodiments

A1. Method of treating or alleviating the symptoms of alcoholic hepatitis in a subject, comprising administering of 2-5 mg canakinumab per kg body weight to the subject.

A2. The method according to embodiment A1, wherein the subject has serum bilirubin levels of >80 μmol/L before administration of canakinumab.

A3. The method according to any of the preceding embodiments, wherein the subject has history of excess alcohol intake characterized by alcohol intake of >80 g/day for males or >60 g/day for females within 6 weeks before administration of canakinumab.

A4. The method according to any of the preceding embodiments, wherein the subject has Maddrey discriminant function (mDF) score of ≥32 before administration of canakinumab.

A5. The method according to any of the preceding embodiments, wherein the subject has Model for End-Stage Liver Disease (MELD) score of ≤25 before administration of canakinumab.

A6. The method according to any of the preceding embodiments, wherein the subject has Maddrey discriminant function (mDF) score of ≥32 and Model for End-Stage Liver Disease (MELD) score of 25 before administration of canakinumab.

A7. The method according to any of the preceding embodiments, wherein the subject has reduced lobular inflammation assessed at least 4 weeks (28 days) from first administration of canakinumab.

A8. The method according to any of the preceding embodiments, wherein the subject has resolution of individual components of alcoholic hepatitis such as polymorphonuclear cell infiltrate, ballooned hepatocytes and/or steatosis in liver cells, wherein the resolution is detected by liver biopsy at least 4 weeks (28 days) after first administration of canakinumab.

A9. The method according to any of the preceding embodiments, wherein antibiotics are administered to said patient for at least 14 days following first administration of canakinumab.

A10. The method according to any of the preceding embodiments, wherein the subject is at high risk of bacterial infection characterized by levels of 16S ribosomal DNA (16S rDNA) in the blood of >18.5 pg/ml and wherein antibiotics are administered for at least 14 days following first administration of canakinumab.

A11. The method according to any of the preceding embodiments, comprising administering at least one additional dose of about 2-5 mg canakinumab per kg body weight, provided said patient has aspartate transaminase (AST) greater than twice upper limit of normal (ULN) assessed at least four weeks (28 days) after initial administration of canakinumab, wherein administration of the initial dose and additional doses are separated in time by at least four weeks (28 days).

A12. The method according to any of the preceding embodiments, wherein 2 mg canakinumab per kg body weight are administered to the subject.

A13. The method according to any of the preceding embodiments, wherein 3 mg canakinumab per kg body weight are administered to the subject.

A14. The method according to any of the preceding embodiments, wherein 4 mg canakinumab per kg body weight are administered to the subject.

A15. The method according to any of the preceding embodiments, wherein 5 mg canakinumab per kg body weight are administered to the subject.

A16. The method according to any of the preceding embodiments, wherein canakinumab is administered parenterally.

A17. The method according to any of the preceding embodiments, wherein canakinumab is administered intravenously or subcutaneously.

A18. The method according to any of the preceding embodiments, wherein canakinumab is administered in a reconstituted formulation comprising canakinumab at a concentration of 10-200 mg/ml, sucrose, histidine and polysorbate 80, wherein the pH of the formulation is 6.1-6.9.

A19. The method according to any of the preceding embodiments, wherein canakinumab is administered intravenously.

A20. The method according to any of embodiments A1-A18, wherein canakinumab is administered subcutaneously.

B1. Method of treating or alleviating the symptoms of alcoholic hepatitis in a subject, comprising administering gevokizumab.

B2. The method according to embodiment B1, wherein the subject has serum bilirubin levels of >80 μmol/L before administration of gevokizumab.

B3. The method according to any of embodiments B1-B2, wherein the subject has history of excess alcohol characterized by alcohol intake of >80 g/day for males or >60 g/day for females within 6 weeks before administration of gevokizumab.

B4. The method according to any of embodiments B1-B3, wherein the subject has Maddrey discriminant function (mDF) score of ≥32 before administration of gevokizumab.

B5. The method according to any of embodiments B1-B4, wherein the subject has Model for End-Stage Liver Disease (MELD) score of ≤25 before administration of gevokizumab.

B6. The method according to any of embodiments B1-B5, wherein the subject has Maddrey discriminant function (mDF) score of ≥32 and Model for End-Stage Liver Disease (MELD) score of 25 before administration of gevokizumab.

B7. The method according to any of embodiments B1-B6, wherein the subject has reduced lobular inflammation assessed at least 4 weeks (28 days) from first administration of gevokizumab.

B8. The method according to any of embodiments B1-B7, wherein the subject has resolution of individual components of alcoholic hepatitis such as polymorphonuclear cell infiltrate, ballooned hepatocytes and/or steatosis in liver cells, wherein the resolution is detected by liver biopsy at least 4 weeks (28 days) after first administration of gevokizumab.

B9. The method according to any of embodiments B1-B8, wherein antibiotics are administered to said patient for at least 14 days following first administration of canakinumab.

B10. The method according to any of embodiments B1-B9, wherein the subject is at high risk of bacterial infection characterized by levels of 16S ribosomal DNA (16S rDNA) in the blood of >18.5 pg/ml and wherein antibiotics are administered for at least 14 days following first administration of gevokizumab.

B11. The method according to any of embodiments B1-B10, comprising administering at least one additional dose of gevokizumab, provided said patient has aspartate transaminase (AST) greater than twice upper limit of normal (ULN) assessed at least four weeks (28 days) after initial administration of gevokizumab, wherein administration of the initial dose and additional doses are separated in time by at least four weeks (28 days).

B12. The method according to any of the preceding embodiments B1-B11, wherein gevokizumab is administered parenterally.

B13. The method according to any of the preceding embodiments B1-B12, wherein gevokizumab is administered intravenously or subcutaneously.

B14. The method according to any of the preceding embodiments B1-B13, wherein gevokizumab is administered intravenously.

B15. The method according to any of the preceding embodiments B1-B13, wherein gevokizumab is administered subcutaneously.

C1. Canakinumab for use in treating or alleviating the symptoms of alcoholic hepatitis in a subject, comprising administering of 2-5 mg canakinumab per kg body weight to the subject.

C2. Use of canakinumab for the manufacture of a medicament for treating or alleviating the symptoms of alcoholic hepatitis in a subject, comprising administering of 2-5 mg canakinumab per kg body weight to the subject.

C3. The use according to any of embodiments C1-C2, wherein the subject has serum bilirubin levels of >80 μmol/L before administration of canakinumab.

C4. The use according to any of embodiments C1-C3, wherein the subject has history of excess alcohol intake characterized by alcohol intake of >80 g/day for males or >60 g/day for females within 6 weeks before administration of canakinumab.

C5. The use according to any of embodiments C1-C4, wherein the subject has Maddrey discriminant function (mDF) score of ≥32 before administration of canakinumab.

C6. The use according to any of embodiments C1-C5, wherein the subject has Model for End-Stage Liver Disease (MELD) score of ≤25 before administration of canakinumab.

C7. The use according to any of embodiments C1-C6, wherein the subject has Maddrey discriminant function (mDF) score of ≥32 and Model for End-Stage Liver Disease (MELD) score of ≤25 before administration of canakinumab.

C8. The use according to any of embodiments C1-C7, wherein the subject has reduced lobular inflammation assessed at least 4 weeks (28 days) from first administration of canakinumab.

C9. The use according to any of embodiments C1-C8, wherein the subject has resolution of individual components of alcoholic hepatitis such as polymorphonuclear cell infiltrate, ballooned hepatocytes and/or steatosis in liver cells, wherein the resolution is detected by liver biopsy at least 4 weeks (28 days) after first administration of canakinumab.

C10. The use according to any of embodiments C1-C9, comprising administering antibiotics for at least 14 days following first administration of canakinumab.

C11. The use according to any of embodiments C1-C10, wherein the subject is at high risk of bacterial infection characterized by levels of 16S ribosomal DNA (16S rDNA) in the blood of >18.5 pg/ml and wherein antibiotics are administered for at least 14 days following first administration of canakinumab.

C12. The use according to any of embodiments C1-C11, comprising administering at least one additional dose of about 2-5 mg canakinumab per kg body weight, provided said patient has aspartate transaminase (AST) greater than twice upper limit of normal (ULN) assessed at least four weeks (28 days) after initial administration of canakinumab, wherein administration of the initial dose and additional doses are separated in time by at least four weeks (28 days).

C13. The use according to any of embodiments C1-C12, wherein 2 mg canakinumab per kg body weight are administered to the subject.

C14. The use according to any of embodiments C1-C13, wherein 3 mg canakinumab per kg body weight are administered to the subject.

C15. The use according to any of embodiments C1-C14, wherein 4 mg canakinumab per kg body weight are administered to the subject.

C16. The use according to any of embodiments C1-C15, wherein 5 mg canakinumab per kg body weight are administered to the subject.

C17. The use according to any of embodiments C1-C16, wherein canakinumab is administered parenterally.

C18. The use according to any of embodiments C1-C17, wherein canakinumab is administered intravenously or subcutaneously.

C19. The use according to embodiment C1-C18, wherein canakinumab is administered in a reconstituted formulation comprising canakinumab at a concentration of 10-200 mg/ml, sucrose, histidine and polysorbate 80, wherein the pH of the formulation is 6.1-6.9.

C20. The use according to any of embodiments C1-C19, wherein canakinumab is administered intravenously.

C21. The use according to any of embodiments C1-C19, wherein canakinumab is administered subcutaneously.

D1. Gevokizumab for use in treating or alleviating the symptoms of alcoholic hepatitis in a subject, comprising administering gevokizumab.

D2. Use of gevokizumab for the manufacture of a medicament for treating or alleviating the symptoms of alcoholic hepatitis in a subject, comprising administering gevokizumab to the subject.

D3. The use according to any of embodiments D1-D2, wherein the subject has serum bilirubin levels of >80 μmol/L before administration of gevokizumab.

D4. The use according to any of embodiments D1-D3, wherein the subject has history of excess alcohol characterized by alcohol intake of >80 g/day for males or >60 g/day for females within 6 weeks before administration of gevokizumab.

D5. The use according to any of embodiments D1-D4, wherein the subject has Maddrey discriminant function (mDF) score of ≥32 before administration of gevokizumab.

D6. The use according to any of embodiments D1-D5, wherein the subject has Maddrey discriminant function (mDF) score of ≥32 and Model for End-Stage Liver Disease (MELD) score of ≤25 before administration of gevokizumab.

D7. The use according to any of embodiments D1-D6, wherein the subject has Maddrey discriminant function (mDF) score of ≥32 and Model for End-Stage Liver Disease (MELD) score of ≤25 before administration of gevokizumab.

D8. The use according to any of embodiments D1-D7, wherein the subject has reduced lobular inflammation assessed at least 4 weeks (28 days) from first administration of gevokizumab.

D9. The use according to any of embodiments D1-D8, wherein the subject has resolution of individual components of alcoholic hepatitis such as polymorphonuclear cell infiltrate, ballooned hepatocytes and/or steatosis in liver cells, wherein the resolution is detected by liver biopsy at least 4 weeks (28 days) after first administration of gevokizumab.

D10. The use according to any of embodiments D1-D9, comprising administering antibiotics for at least 14 days following first administration of gevokizumab.

D11. The use according to any of embodiments D1-D10, wherein the subject is at high risk of bacterial infection characterized by levels of 16S ribosomal DNA (16S rDNA) in the blood of >18.5 pg/ml and wherein antibiotics are administered for at least 14 days following first administration of gevokizumab.

D12. The use according to any of embodiments D1-D11, comprising administering at least one additional dose of gevokizumab, provided said patient has aspartate transaminase (AST) greater than twice upper limit of normal (ULN) assessed at least four weeks (28 days) after initial administration of gevokizumab, wherein administration of the initial dose and additional doses are separated in time by at least four weeks (28 days).

D13. The use according to any of embodiments D1-D12, wherein gevokizumab is administered parenterally.

D14. The use according to any of embodiments D1-D13, wherein gevokizumab is administered subcutaneously or intravenously.

D15. The use according to any of embodiments D1-D14, wherein gevokizumab is administered intravenously.

D16. The use according to any of embodiments D1-D14, wherein gevokizumab is administered subcutaneously.

E1. A pharmaceutical composition comprising canakinumab and at least one pharmaceutically acceptable carrier, diluent or excipient for use in treating or alleviating the symptoms of alcoholic hepatitis in a subject, comprising administering of 2-5 mg canakinumab per kg body weight to the subject.

E2. The pharmaceutical composition for use according to embodiment E1, wherein the subject has serum bilirubin levels of >80 μmol/L before administration of canakinumab.

E4. The pharmaceutical composition for use according to any of embodiments E1-E3, wherein the subject has history of excess alcohol intake characterized by alcohol intake of >80 g/day for males or >60 g/day for females within 6 weeks before administration of canakinumab.

E5. The pharmaceutical composition for use according to any of embodiments E1-E4, wherein the subject has Maddrey discriminant function (mDF) score of ≥32 before administration of canakinumab.

E6. The pharmaceutical composition for use according to any of embodiments E1-E5, wherein the subject has Model for End-Stage Liver Disease (MELD) score of ≤25 before administration of canakinumab.

E7. The pharmaceutical composition for use according to any of embodiments E1-E6, wherein the subject has Maddrey discriminant function (mDF) score of ≥32 and Model for End-Stage Liver Disease (MELD) score of ≤25 before administration of canakinumab.

E8. The pharmaceutical composition for use according to any of embodiments E1-E7, wherein the subject has reduced lobular inflammation assessed at least 4 weeks (28 days) from first administration of canakinumab.

E9. The pharmaceutical composition for use according to any of embodiments E1-E8, wherein the subject has resolution of individual components of alcoholic hepatitis such as polymorphonuclear cell infiltrate, ballooned hepatocytes and/or steatosis in liver cells, wherein the resolution is detected by liver biopsy at least 4 weeks (28 days) after first administration of canakinumab.

E10. The pharmaceutical composition for use according to any of embodiments E1-E9, comprising administering antibiotics for at least 14 days following first administration of canakinumab.

E11. The pharmaceutical composition for use according to any of embodiments E1-E10, wherein the subject is at high risk of bacterial infection characterized by levels of 16S ribosomal DNA (16S rDNA) in the blood of >18.5 pg/ml and wherein antibiotics are administered for at least 14 days following first administration of canakinumab.

E12. The pharmaceutical composition for use according to any of embodiments E1-E11, comprising administering at least one additional dose of about 2-5 mg canakinumab per kg body weight, provided said patient has aspartate transaminase (AST) greater than twice upper limit of normal (ULN) assessed at least four weeks (28 days) after initial administration of canakinumab, wherein administration of the initial dose and additional doses are separated in time by at least four weeks (28 days).

E13. The pharmaceutical composition for use according to any of embodiments E1-E12, wherein 2 mg canakinumab per kg body weight are administered to the subject.

E14. The pharmaceutical composition for use according to any of embodiments E1-E13, wherein 3 mg canakinumab per kg body weight are administered to the subject.

E15. The pharmaceutical composition for use according to any of embodiments E1-E14, wherein 4 mg canakinumab per kg body weight are administered to the subject.

E16. The pharmaceutical composition for use according to any of embodiments E1-E15, wherein 5 mg canakinumab per kg body weight are administered to the subject.

E17. The pharmaceutical composition for use according to any of embodiments E1-E16, wherein canakinumab is administered parenterally.

E18. The pharmaceutical composition for use according to any of embodiments E1-E17, wherein canakinumab is administered intravenously or subcutaneously.

E19. The pharmaceutical composition for use according to embodiment E1-E18, wherein canakinumab is administered in a reconstituted formulation comprising canakinumab at a concentration of 10-200 mg/ml, sucrose, histidine and polysorbate 80, wherein the pH of the formulation is 6.1-6.9.

E20. The pharmaceutical composition for use according to any of embodiments E1-E19, wherein canakinumab is administered intravenously.

E21. The pharmaceutical composition for use according to any of embodiments E1-E19, wherein canakinumab is administered subcutaneously.

F1. A pharmaceutical composition comprising gevokizumab and at least one pharmaceutically acceptable carrier, diluent or excipient for use in treating or alleviating the symptoms of alcoholic hepatitis in a subject, comprising administering gevokizumab.

F2. The pharmaceutical composition for use according to embodiment F1, wherein the subject has serum bilirubin levels of >80 μmol/L before administration of gevokizumab.

F3. The pharmaceutical composition for use according to any of embodiments F1-F2, wherein the subject has history of excess alcohol characterized by alcohol intake of >80 g/day for males or >60 g/day for females within 6 weeks before administration of gevokizumab.

F4. The pharmaceutical composition for use according to any of embodiments F1-F3, wherein the subject has Maddrey discriminant function (mDF) score of ≥32 before administration of gevokizumab.

F5. The pharmaceutical composition for use according to any of embodiments F1-F4, wherein the subject has Maddrey discriminant function (mDF) score of ≥32 and Model for End-Stage Liver Disease (MELD) score of ≤25 before administration of gevokizumab.

F6. The pharmaceutical composition for use according to any of embodiments F1-F5, wherein the subject has Maddrey discriminant function (mDF) score of ≥32 and Model for End-Stage Liver Disease (MELD) score of ≤25 before administration of gevokizumab.

F7. The pharmaceutical composition for use according to any of embodiments F1-F6, wherein the subject has reduced lobular inflammation assessed at least 4 weeks (28 days) from first administration of gevokizumab.

F8. The pharmaceutical composition for use according to any of embodiments F1-F7, wherein the subject has resolution of individual components of alcoholic hepatitis such as polymorphonuclear cell infiltrate, ballooned hepatocytes and/or steatosis in liver cells, wherein the resolution is detected by liver biopsy at least 4 weeks (28 days) after first administration of gevokizumab.

F9. The pharmaceutical composition for use according to any of embodiments F1-F8, comprising administering antibiotics for at least 14 days following first administration of gevokizumab.

F10. The pharmaceutical composition for use according to any of embodiments F1-F9, wherein the subject is at high risk of bacterial infection characterized by levels of 16S ribosomal DNA (16S rDNA) in the blood of >18.5 pg/ml and wherein antibiotics are administered for at least 14 days following first administration of gevokizumab.

F11. The pharmaceutical composition for use according to any of embodiments F1-F10, comprising administering at least one additional dose of gevokizumab, provided said patient has aspartate transaminase (AST) greater than twice upper limit of normal (ULN) assessed at least four weeks (28 days) after initial administration of gevokizumab, wherein administration of the initial dose and additional doses are separated in time by at least four weeks (28 days).

F12. The pharmaceutical composition for use according to any of embodiments F1-F11, wherein gevokizumab is administered parenterally.

F13. The pharmaceutical composition for use according to any of embodiments F1-F12, wherein gevokizumab is administered subcutaneously or intravenously.

F14. The pharmaceutical composition for use according to any of embodiments F1-F13, wherein gevokizumab is administered intravenously.

F15. The pharmaceutical composition for use according to any of embodiments F1-F13, wherein gevokizumab is administered subcutaneously.

The skilled person realizes that the features, aspects and embodiments taught in the text are all combinable with each other and particular aspects combining features and/or embodiments from various parts of the text will be considered to be adequately disclosed to the skilled person.

Additional embodiments include pharmaceutical compositions and methods of the uses set forth above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### General:

All patents, published patent applications, publications, references and other material referred to herein are incorporated by reference herein in their entirety.

As used herein, the terms “a” and “an” and “the” and similar references in the context of describing the invention are to be construed to cover both the singular and the plural, unless otherwise indicated herein or clearly contradicted by context.

The term “or” is used herein to mean, and is used interchangeably with the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When describing a dosage herein as “about” a specified amount, the actual dosage can vary by up to 10% from the stated amount: this usage of “about” recognizes that the precise amount in a given dosage form may differ slightly from an intended amount for various reasons without materially affecting the in vivo effect of the administered compound.

As used herein, the term “4 weeks (28 days)” includes a time period that extends three days before and three days after the 4 weeks (4 weeks+/−3 days or 28 days+/−3 days).

As used herein, the term “comprising” encompasses “including” as well as “consisting,” e.g. a composition “comprising” X may consist exclusively of X or may include something additional, e.g., X+Y.

As used herein, the term “administering” in relation to a compound, e.g., an IL-1β binding antibody such as canakinumab or gevokizumab, is used to refer to delivery of that compound by any route of delivery, e.g. parenterally, e.g. subcutaneously or intravenously, to a subject in need thereof.

As used herein, the term “patient” and “subject” includes any human patient or human subject can be used interchangeably. In one embodiment, the subject is a human, e.g. a human suffering from suffering from alcoholic hepatitis. In another embodiment, said saubject is suffering from acute alcoholic hepatitis and/or severe alcoholic hepatitis. Said patient may have been hospitalized for acute alcoholic hepatitis and/or severe alcoholic hepatitis.

As used herein, a subject is “in need of” a treatment if such subject (patient) would benefit biologically, medically or in quality of life from such treatment.

As used herein, the term “baseline” denotes a given parameter or the state of the patient before administration of canakinumab or before administration of gevokizumab.

As used herein, “treating” or “treat” describes the management and care of a patient for the purpose of combating a disease, condition, or disorder and includes the administration of canakinumab or gevokizumab to alleviate the symptoms or complications of a disease, condition or disorder, or to eliminate the disease, condition or disorder.

As used herein, the term “alleviate” or “alleviating” is meant to describe a process by which the severity of a sign or symptom of a disorder is decreased. Importantly, a sign or symptom can be alleviated without being eliminated. The administration of canakinumab or gevokizumab may or can lead to the elimination of a sign or symptom, however, elimination is not required. Effective dosages should be expected to decrease the severity of a sign or symptom.

As used herein, the term “pharmaceutically acceptable carrier, diluent or excipient” or “carrier, diluent or excipient” refers to a substance useful in the preparation or use of a pharmaceutical composition, which enhance or stabilize the composition, or can be used to facilitate the preparation of the composition. Pharmaceutically acceptable carriers include solvents, dispersion media, coatings, antibacterial and antifungal agents, isotonic and absorption delaying agents, and the like, and includes, for example, suitable diluents, surfactants, antioxidants, preservatives, buffering agents, emulsifiers, salts, drug stabilizers, binders, excipients, disintegration agents, lubricants, wetting agents, sweetening agents, flavoring agents, dyes, and combinations thereof, solvents, dispersion media, coatings, antibacterial and antifungal agents, isotonic and absorption delaying agents, and the like that are physiologically compatible and that do not produce an adverse, allergic or other untoward reaction when administered to an animal, or a human, as appropriate, as would be known to those skilled in the art (see, for example, Remington The Science and Practice of Pharmacy, 22^{nd} Ed. Pharmaceutical Press, 2013, pp. 1049-1070). Except insofar as any conventional media or agent is incompatible with the active compound, use thereof in the compositions described herein is contemplated.

The term “pharmaceutical composition” as used herein, refers to an IL-1β binding antibody, e.g., canakinumab or gevokizumab, together with at least one pharmaceutically acceptable carrier, in a form suitable for administration to the physical location most suitable for their desired activity, e.g., systemic administration. The term “pharmaceutical composition” refers to a mixture or solution containing at least one therapeutic agent, preferably an an IL-1β binding antibody, e.g., canakinumab or gevokizumab, to be administered to a subject, e.g. a mammal or human, in order or treat a particular disease, e.g. alcoholic hepatitis, affecting the subject.

Without intending to limit the scope of the invention in any way, it is further described by way of illustration of the following example.

### EXAMPLE

A multicenter, double blind, randomized (1:1), placebo controlled trial evaluating the efficacy, safety and tolerability of canakinumab in patients with alcoholic hepatitis

### Inclusion Criteria

Alcoholic hepatitis patients eligible for inclusion in this study must fulfill all of the following criteria:
Male and female patients aged 18 years or older at screening
Clinical alcoholic hepatitis at screening:
   Serum bilirubin>80 μmol/L
   History of excess alcohol (>80 g/day male, >60 g/day female) to within 6 weeks before screening visit
Less than 4 weeks since admission to hospital at baseline visit
mDF≥32 and MELD≤25 at baseline visit
Written informed consent must be obtained before any assessment is performed.
Women of child-bearing potential have to use an effective contraception method

### Exclusion Criteria

Alcohol abstinence of >6 weeks prior to randomization/baseline visit
Duration of clinically apparent jaundice>3 months before baseline visit
Other causes of liver disease including:
   Evidence of chronic viral hepatitis (Hepatitis B or C)
   Biliary obstruction
   Hepatocellular carcinoma
Evidence of current malignancy (except non-melanotic skin cancer)
Previous entry into the study, or use of either prednisolone or pentoxifylline (PTX) within 6 weeks of hospital admission
AST>500 U/L or ALT>300 U/L (not compatible with alcoholic hepatitis)
Patients with a serum creatinine>220 μmol/L (2.5 mg/dL) or requiring renal support (Patients who are oligo-anuric, have a creatinine>220 μmol/L (2.5 mg/dL) or who require renal support, will be given appropriate resuscitation therapy for up to 1 week. These patients may then be re-screened and considered for randomisation, once they meet eligibility criteria.)
Patients dependent upon inotropic support (adrenaline or noradrenaline). Terlipressin is allowed
Variceal hemorrhage on admission
Untreated sepsis

### Sepsis

As per standard of care all patients are screened for infection prior to randomization. Diagnosis of infection is based on the criteria outlined by Bajaj et al. and involves chest radiography, urinalysis (mid-stream urine (MSU) culture if urinalysis positive), ascitic tap (if ascites present) and blood cultures if pyrexial. Positive culture and initiation of antibiotics with clinical or radiological signs of infection, as well as clinical suspicion, are recorded as sepsis.

Blood culture negative pyrexia and a leucocytosis are not regarded as signs of active sepsis on their own, as these are often co-existent findings with alcoholic hepatitis. Patients with evidence of sepsis are treated for a minimum of 2 days with appropriate antibiotics before re-screening.

Once the local Principal Investigator (PI) considers that the sepsis is under control, the patient may be re-screened and randomised if eligible. Patients with baseline infection should continue antibiotic treatment for 2 weeks after initiation of treatment.

Bacterial DNA is measured on whole blood samples (EDTA tube) at screening. Patients found to have bacterial DNA (16S rDNA)>18.5 pg/ml are treated with prophylactic antibiotics (co-amoxyclav or ciprofloxacin) for the first 14 days treatment irrespective of whether they are randomized to canakinumab or placebo. Patients are screened for infection at baseline and on a weekly basis.

### Treatment

Patients are included and randomized and treated before histology result is available. If the histology is negative then patient is withdrawn.

A single dose of 3 mg/kg Canakinumab or identical placebo is administered intravenously at baseline (Day 1).

Patients with AST>2×ULN on Day 28 receive a second dose of 3 mg/kg study drug administered intravenously (i.v.) on Day 28:
Patients who received placebo on baseline receive placebo.
Patients who received canakinumab on baseline receive canakinumab.

### Primary End Point

Histological improvement of alcoholic hepatitis on liver biopsy 28 days after initial administration of canakinumab compared to baseline. Histological improvement is defined as reduction in lobular inflammation, regardless of cell type.

### Secondary End Points

Resolution of individual components (polymorphonuclear cell infiltrate, ballooned hepatocytes and/or steatosis) of alcoholic hepatitis on liver histology from baseline to Day 28
Changes in the components of Alcoholic Hepatitis Histological Score (AHHS) (Altamirano et al (2014) Gastroenterology., 146(5), 1231-9.e1-6) from baseline to Day 28
Changes in the components of Nonalcoholic fatty liver disease activity score (NAS) score from baseline to Day 28
Changes in hepatic venous pressure gradient (HVPG) between baseline and day 28
Changes of serum CK18-M30/M65 from baseline to Day 7, 14, 21, 28, 42 and 90
Change in serum bilirubin from baseline to Day 7, 14, 28, 21, 42 and 90
Change in MELD score at from baseline to Day 7, 14, 21, 28, 42 and 90
Change in Glasgow alcoholic hepatitis score (GAHS) from baseline to Day 7, 14, 21, 28, 42 and 90
Change in mDF score from baseline to Day 7, 14, 21, 28, 42 and 90
Lille score at Day 7
Resolution of systemic inflammatory response syndrome (SIRS) at Day 7, 14, 21, 28, 42 and 90 in patients with SIRS at baseline
   SIRS according to the Recommendations of the American College of Chest Physicians/Society of Critical Care Medicine Consensus Conference, wherein presence of 2 or more criteria out of following are required:
      Temperature<36° C. or >38° C.
      Heart rate>90 beats/minute
      Respiratory rate>20 breaths/minute or venous pCO2<32 mmHg
      Leukocyte count>12,000/mm3 or <4,000/mm3 or band forms>10%
Incidence of SIRS at Day 7, 14, 21, 28, 42 and 90 in patients without SIRS at baseline
Mortality rate at Day 90
Incidence of infection and sepsis over 90 days
Incidence of acute kidney injury over 90 days
Incidence of variceal hemorrhage, ascites or encephalopathy over 90 days

1. A Method of treating or alleviating the symptoms of alcoholic hepatitis in a subject, comprising administering of 2-5 mg canakinumab per kg body weight to the subject.

2. The method according to claim 1, wherein the subject has serum bilirubin levels of >80 μmol/L before administration of canakinumab.

3. The method according to claim 1 or 2, wherein the subject has history of excess alcohol characterized by alcohol intake of >80 g/day for males or >60 g/day for females within 6 weeks before administration of canakinumab.

4. The method according to any of the preceding claims, wherein the subject has Maddrey discriminant function (mDF) score of ≥32 and Model for End-Stage Liver Disease (MELD) score of ≤25 before administration of canakinumab.

5. The method according to any of the preceding claims, comprising administering at least one additional dose of about 2-5 mg canakinumab per kg body weight, provided said patient has aspartate transaminase (AST) greater than twice upper limit of normal (ULN) assessed at least four weeks (28 days) after initial administration of canakinumab, and wherein administration of the initial dose and additional doses are separated in time by at least four weeks (28 days).

6. The method according to any of the preceding claims, wherein 3 mg per kg body weight of canakinumab are administered.

7. The method according to any of the preceding claims, wherein canakinumab is administered parenterally, suitably intravenously.

8. The method according to claim 7, wherein canakinumab is administered in a reconstituted formulation comprising canakinumab at a concentration of 10-200 mg/ml, sucrose, histidine and polysorbate 80, wherein the pH of the formulation is 6.1-6.9.

9. A Method of treating or alleviating the symptoms of alcoholic hepatitis in a subject, comprising administering gevokizumab.

10. The method according to claim 9, wherein the subject has serum bilirubin levels of >80 μmol/L before administration of gevokizumab.

11. The method according to any claim 9 or 10, wherein the subject has history of excess alcohol characterized by alcohol intake of >80 g/day for males or >60 g/day for females within 6 weeks before administration of gevokizumab.

12. The method according to any of claims 9-11, wherein the subject has Maddrey discriminant function (mDF) score of ≥32 and Model for End-Stage Liver Disease (MELD) score of ≤25 before administration of gevokizumab.

13. The method according to any of claims 9-12, comprising administering at least one additional dose of gevokizumab, provided said patient has aspartate transaminase (AST) greater than twice upper limit of normal (ULN) assessed at least four weeks (28 days) after initial administration of gevokizumab, wherein administration of the initial dose and additional doses are separated in time by at least four weeks (28 days).

14. The method according to any of claims 9-13, wherein gevokizumab is administered parenterally.

15. Use of canakinumab in treating or alleviating the symptoms of alcoholic hepatitis in a subject, comprising administering of 2-5 mg canakinumab per kg body weight to the subject.

16. Use of canakinumab for the manufacture of a medicament for treating or alleviating the symptoms of alcoholic hepatitis in a subject, comprising administering of 2-5 mg canakinumab per kg body weight to the subject.

17. The use according to any of claims 15-16, wherein the subject has serum bilirubin levels of >80 μmol/L before administration of canakinumab.

18. The use according to any of claims 15-17, wherein the subject has history of excess alcohol characterized by alcohol intake of >80 g/day for males or >60 g/day for females within 6 weeks before administration of canakinumab.

19. The use according to any of claims 15-18, wherein the subject has Maddrey discriminant function (mDF) score of ≥32 and Model for End-Stage Liver Disease (MELD) score of ≤25 before administration of canakinumab.

20. The use according to any of claims 15-19, comprising administering at least one additional dose of about 2-5 mg canakinumab per kg body weight, provided said patient has aspartate transaminase (AST) greater than twice upper limit of normal (ULN) assessed at least four weeks (28 days) after initial administration of canakinumab, wherein administration of the initial dose and additional doses are separated in time by at least four weeks (28 days).

21. The use according to any of claims 15-20, wherein 3 mg canakinumab per kg body weight are administered to the subject.

22. The use according to any of claims 15-21, wherein canakinumab is administered parenterally, suitably intravenously.

23. The method according to claim 15-22, wherein canakinumab is administered in a reconstituted formulation comprising canakinumab at a concentration of 10-200 mg/ml, sucrose, histidine and polysorbate 80, wherein the pH of the formulation is 6.1-6.9.

24. Use of gevokizumab in treating or alleviating the symptoms of alcoholic hepatitis in a subject, comprising administering gevokizumab.

25. Use of gevokizumab for the manufacture of a medicament for treating or alleviating the symptoms of alcoholic hepatitis in a subject, comprising administering gevokizumab to the subject.

26. The use according to any of claims 24-25, wherein the subject has serum bilirubin levels of >80 μmol/L before administration of gevokizumab.

27. The use according to any of claims 24-26, wherein the subject has history of excess alcohol characterized by alcohol intake of >80 g/day for males or >60 g/day for females within 6 weeks before administration of gevokizumab.

28. The use according to any of claims 24-27, wherein the subject has Maddrey discriminant function (mDF) score of ≥32 and Model for End-Stage Liver Disease (MELD) score of ≤25 before administration of gevokizumab.

29. The use according to any of claims 24-28, comprising administering at least one additional dose of gevokizumab, provided said patient has aspartate transaminase (AST) greater than twice upper limit of normal (ULN) assessed at least four weeks (28 days) after initial administration of gevokizumab, wherein administration of the initial dose and additional doses are separated in time by at least four weeks (28 days).

30. The use according to any of claims 24-29, wherein gevokizumab is administered parenterally.
